(19) United States
(12) Patent Application Publication
(10) Pub. No.: US 2017/0246100 A1
(43) Pub. Date: Aug. 31, 2017

(54) AGENT FOR TEMPORARILY RESHAPING KERATIN-CONTAINING FIBERS COMPRISING A POLYMER MIXTURE

(71) Applicant: Henkel AG & Co. KGaA, Duesseldorf (DE)

(72) Inventor: Anna Puls, Winsen (Luhe) (DE)

(73) Assignee: Henkel AG & Co. KGaA, Duesseldorf (DE)

(21) Appl. No.: 15/444,019

(22) Filed: Feb. 27, 2017

(30) Foreign Application Priority Data
Feb. 29, 2016 (DE) 10 2016 203 231.3

(51) Int. Cl. A61K 8/84; A61Q 5/06; A61K 8/81

(52) U.S. Cl. CPC A61K 8/84; A61K 8/81; A61Q 5/06; A61K 2800/48; A61K 2800/594

(57) ABSTRACT

Transparent cosmetic compositions include, based on the total weight thereof,
a) 0.1 to 10 wt. % of a conditioning polymer;
b) 0.5 to 25 wt. % of a thickening polymer;
c) 0.01 to 30 wt. % of a film-forming polymer;
d) 1 to 35 wt. % of a humectant;
e) 0.1 to 6 wt. % of an acid, and
f) 1 to 90 wt. % water.

The cosmetic compositions are particularly suitable for temporarily reshaping keratin fibers, and in particular curly human hair.

### FIELD OF THE INVENTION

This invention relates to the technical field of temporarily reshaping keratin-containing fibers, and in particular human hair.

### BACKGROUND OF THE INVENTION

Styling agents for reshaping keratin-containing fibers have been known for quite some time and are used in a variety of forms to create, revive and impart hold to hair styles that, in many hair types, can only be achieved by using setting active ingredients. In this process, hair treatment agents that are used to impart shape to the hair both permanently and temporarily play an important role.

Sprayable products as well as product forms that are worked into the hair style by way of a comb or the fingers are suitable for imparting a temporary shape to keratin-containing fibers. The product group mentioned last comprises oils as well as gels, creams and powders.

Frizzy hair is one of the biggest problems and occurs when the hair is exposed to elevated moisture in the air. This problem typically occurs to a greater degree in people who have curls and results in what colloquially is referred to as a “bad hair day.” In such a case, the hair loses the natural shape and/or the curl retention thereof

People with naturally curly hair or perms additionally often encounter the problem that the curls begin to sag over time and lose their bounce. This takes place, for example, when the hair treatment agents used, such as shampoos, rinse conditioners or styling agents, weigh down the hair.

Unlike straight hair, curly hair does not reflect as much light, whereby the hair is also less shiny than it would be customarily.

EP1741470 B1 describes a composition to improve shine, manageability, curl retention and curl separation of curly hair. This composition comprises a film-forming polymer, at least 5 wt. % polyol, and polyacryloyldimethyl taurate and/or the salts thereof

Another problem in formulating styling agents is to produce clear, transparent compositions since the polymers typically used in hair styling agents often yield milky/cloudy compositions. A milky appearance, however, may give the user the impression that the formulation could be clearly visible in the hair, thereby reducing the customer's acceptance of the styling agent.

It is therefore desirable to provide transparent hair treatment agents for temporarily shaping curly hair, which impart a lasting, curly appearance, high volume, high shine, and good manageability to the hair.

A lasting, curly appearance is marked by high curl retention and/or high bounce of the curls and/or high curl separation. The latter means that the curls can be clearly identified in a strand of hair.

Other desirable features and characteristics of the present invention will become apparent from the subsequent detailed description of the invention and the appended claims, taken in conjunction with this background of the invention.

### BRIEF SUMMARY OF THE INVENTION

According to one embodiment, the present invention is a transparent cosmetic composition, comprising, based on the total weight thereof:
a) 0.1 to 10 wt. % of a conditioning polymer;
b) 0.5 to 25 wt. % of a thickening polymer;
c) 0.01 to 30 wt. % of a film-forming polymer;
d) 1 to 35 wt. % of a humectant;
e) 0.1 to 6 wt. % of an acid, and
f) 1 to 90 wt. % water.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was found that this object can be achieved by a combination of a conditioning polymer, a thickening polymer, a film-forming polymer, a humectant and an acid.

The composition is present in the form of a gel, which is preferably applied directly using the hand or a comb, in the form of a viscous lotion, cream or paste, in the form of a wax, in the form of a sprayable liquid sprayed by way of a mechanical device, or in the form of a foamable liquid foamed prior to application. The cosmetic composition is preferably present in the form of a gel.

Corresponding hair treatment agents are referred to as hair sprays, hair gel, hair wax, hair foam, hair setting lotion or hair lotion. Clay denotes a further possible product form. This refers to high viscosity, wax-like cosmetic compositions containing clay compounds (such as kaolin), among other things. Hair gels are preferred cosmetic compositions.

If the compositions are present in the form of gels, creams, pastes or waxes, the viscosity of the compositions is especially between 5,000 and 150,000 mPas, and preferably between 20,000 and 80,000 mPas (20° C., DV 2T “Brookfield” rotational viscometer, spindle 5, 5 revolutions per minute and with Helipath).

Clear transparent compositions within the scope of the present invention shall be understood to mean compositions that have a clear appearance to the naked eye on the palm of the hand (hand clear). A transmission measurement at 600 nm and room temperature showed these compositions to have a transmission value of >93%.

A first essential component of the cosmetic composition is a conditioning polymer.

Conditioning polymers are polymers that attach to the hair and exert a conditioning effect there. To improve the attachment behavior, conditioning polymers generally have cationic charges and accordingly are cationic or amphoteric polymers.

The conditioning polymers can be homopolymers or copolymers or polymers based on natural or synthetic polymers. The conditioning polymers can be cationic or amphoteric. The conditioning polymers comprise quaternary nitrogen groups, which are present either in the polymer chain, or preferably as substituents on one or more of the monomers. Suitable cationic monomers are unsaturated, radically polymerizable compounds carrying at least one cationic group, in particular ammonium-substituted vinyl monomers, such as trialkyl methacryloxy alkylammonium, trialkyl acryloxy alkylammonium, dialkyl diallyl ammonium, and quaternary vinyl ammonium monomers comprising cyclic, cationic nitrogen-containing groups, such as pyridinium, imidazolium, or quaternary pyrrolidones, such as alkyl vinylimidazolium, alkyl vinylpyridinium or alkyl vinylpyrrolidone salts. The alkyl groups of these monomers are preferably low alkyl groups, such as C1 to C7 alkyl groups, and particularly preferably C1 to C3 alkyl groups.

The ammonium group-comprising monomers can be co-polymerized with non-cationic monomers. Suitable comonomers are, for example, acrylamide, methacrylamide, alkyl and dialkyl acrylamide, alkyl and dialkyl methacrylamide, alkyl acrylate, alkyl methacrylate, vinyl caprolactone, vinylcaprolactam, vinylpyrrolidone, vinyl ester, such as vinyl acetate, vinyl alcohol, propylene glycol or ethylene glycol, wherein the alkyl groups of these monomers are preferably C1 to C7 alkyl groups, and particularly preferably C1 to C3 alkyl groups.

Out of the multitude of these polymers, the following have proven to be particularly: homopolymers of general formula −{CH₂—[CR¹COO—(CH₂—), N⁺R²R³R⁴]}. X⁻, in which R¹=—H or —CH₃, R², R³ and R⁴, independently of one another, are selected from C1-4 alkyl, -alkenyl- or -hydroxyalkyl groups, m=1, 2, 3 or 4, n is a natural number, and X⁻ is a physiologically compatible organic or inorganic anion. Among these polymers, those to which at least one of the following conditions applies are preferred: R¹ denotes a methyl group, R², R³ and R⁴ denote methyl groups, m has the value 2.

Suitable physiologically compatible counterions X⁻ are, for example, halide ions, sulfate ions, phosphate ions, methosulfate ions, and organic ions, such as lactate, citrate, tartrate and acetate ions. Methosulfates and halide ions, and in particular chloride, are preferred.

Suitable cationic, conditioning polymers are, for example, copolymers according to formula (Copo)

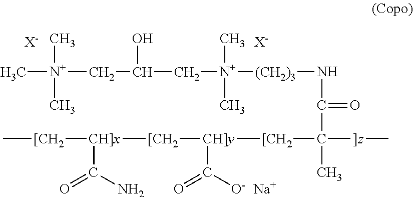

in which the following applies:

x+y+z= Q
Q denotes values from 3 to 55000, especially from 10 to 25000, particularly preferably from 50 to 15000, more preferably from 100 to 10000, still more preferably from 500 to 8000, and in particular from 1000 to 5000;
x denotes (0 to 0.5) Q, especially (0 to 0.3) Q, and in particular the values 0, 1, 2, 3, 4, 5, the value 0 being preferred;
y denotes (0.1 to 0.95) Q, especially (0.5 to 0.7) Q, and in particular values from 1 to 24000, especially from 5 to 15000, particularly preferably from 10 to 10000, and in particular from 100 to 4800;
z denotes (0.001 to 0.5) Q, especially (0.1 to 0.5) Q, and in particular values from 1 to 12500, especially from 2 to 8000, particularly preferably from 3 to 4000, and in particular from 5 to 2000.

Regardless of which of the preferred copolymers of formula (Copo) are used, cosmetic compositions that are characterized in that the ratio of (y:z) is 4:1 to 1:2, and especially 4:1 to 1:1, are preferred.

Regardless of which copolymers are used in the compositions, preferred cosmetic compositions are those in which the copolymer has a molar mass of 10000 to 20 million gmol⁻¹, especially of 100000 to 10 million gmol⁻¹, more preferably of 500000 to 5 million gmol⁻¹ and in particular of 1.1 million to 2.2 million gmol⁻¹.

A most preferred copolymer, which has the composition as described above, is commercially available under the designation Polyquaternium-74.

Suitable cationic, conditioning polymers that are derived from natural polymers are cationic derivatives of polysaccharides, for example cationic derivatives of cellulose, guar or starch. Furthermore, chitosan and chitosan derivatives are suited.
Cationic polysaccharides have the general formula G—O—B—N+RₐR_{b}R_{c}A⁻
G is an anhydroglucose unit, for example starch or cellulose anhydroglucose;
B is a divalent linkage group, such as alkylene, oxyalkylene, polyoxyalkylene or hydroxyalkylene;
Rₐ, R_{b} and R_{c}, independently of one another, are alkyl, aryl, alkylaryl, arylalkyl, alkoxyalkyl or alkoxyaryl, each having up to 18 carbon atoms, wherein the total number of carbon atoms in Rₐ, R_{b} and R, preferably does not exceed 20;
A⁻ is a conventional counterion and preferably is chloride.

Cationic, which is to say quaternized, celluloses are available in the market with differing degrees of substitution, cationic charge densities, nitrogen contents and molecular weights. For example, Polyquaternium-67 is commercially available under the designations SoftCat ®Polymer SL or SoftCat® Polymer SK (Dow).

Further cationic celluloses are Ucare® Polymer JR 400 (Dow, INCI name Polyquaternium-10) and Polymer Quatrisoft° LM-200 (Dow, INCI name Polyquaternium-24). Further commercial products are the compounds Celquat® H 100 and Celquat® L 200.

A suitable chitosan is sold by Kyowa Oil & Fat, Japan, for example, under the trade name Flonac®. Chitosonium pyrrolidone carboxylate, which is sold under the designation Kytamer® PC by Amerchol, USA, for example, is a preferred chitosan salt. Further chitosan derivatives are freely commercially available under the trade names Hydagen® CMF, Hydagen® HCMF and Chitolam® NB/101.

Another group of cationic, conditioning polymers that is excellent to use is glucose-based polymers. The following illustration shows such a cationic alkyl oligoglucoside.

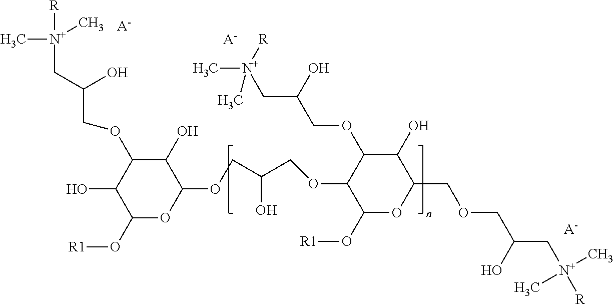

In the above-shown formula, the groups R, independently of one another, denote a linear or branched C6 to C30 alkyl group, a linear or branched C6 to C30 alkenyl group, and preferably the group R denotes a group R selected from: lauryl, myristyl, cetyl, stearyl, oleyl, behenyl or arachidyl.

The groups R1, independently of one another, denote a linear or branched C6 to C30 alkyl group, a linear or branched C6 to C30 alkenyl group, and the group R especially denotes a group selected from: butyl, capryl, caprylyl, octyl, nonyl, decanyl, lauryl, myristyl, cetyl, stearyl, oleyl, behenyl or arachidyl. It is particularly preferred if the groups R1 are identical. Still more preferably, the groups R1 are selected from technical mixtures of the fatty alcohol cuts from C6/C8 fatty alcohols, C8/C10 fatty alcohols, C10/C12 fatty alcohols, C12/C14 fatty alcohols, C12/C18 fatty alcohols, and most preferably those technical fatty alcohol cuts that are of plant origin. The counterion for the cationic charge is a physiologically compatible anion, such as halide, methosulfate, phosphate, citrate, tartrate and the like. The counterion is preferably a halide, such as fluoride, chloride, bromide or methosulfate. Most preferably, the anion is chloride.

Particularly preferred examples of the cationic alkyl oligoglucosides are the compounds with the INCI names Polyquaternium-77, Polyquaternium-78, Polyquaternium-79, Polyquaternium-80, Polyquaternium-81 and Polyquaternium-82. Most preferred are the cationic alkyl oligoglucosides with the INCI names Polyquaternium-77, Polyquaternium-81 and Polyquaternium-82.

Such compounds can be obtained from Colonial Chemical Inc. under the designation Poly Suga® Quat, for example.

This also covers, of course, that multiple mixtures of cationic alkyl oligoglucosides can be used. It is preferred in this case if a long-chain and a short-chain cationic alkyl oligoglucoside are used simultaneously.

A further preferred cationic, conditioning polymer may be obtained based on ethanolamine. The polymer is commercially available under the designation Polyquaternium-71.

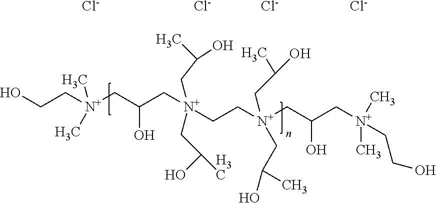

This polymer can be obtained from Colonial Chemical Inc. under the designation Cola® Moist 300 P, for example.

Furthermore, particularly preferably a cationic alkyl oligoglucoside, as shown in the following illustration, can be used.

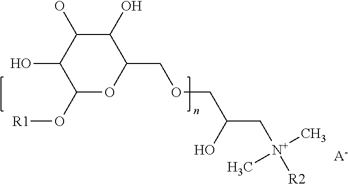

In the above-shown formula, the group R2 denotes a linear or branched C6 to C30 alkyl group, a linear or branched C6 to C30 alkenyl group, and preferably the group R denotes a group R selected from: lauryl, myristyl, cetyl, stearyl, oleyl, behenyl or arachidyl.

The group R1 denotes a linear or branched C6 to C30 alkyl group, a linear or branched C6 to C30 alkenyl group, and preferably the group R1 denotes a group selected from: butyl, capryl, caprylyl, octyl, nonyl, decanyl, lauryl, myristyl, cetyl, stearyl, oleyl, behenyl or arachidyl. Still more preferably, the group R1 is selected from technical mixtures of the fatty alcohol cuts from C6/C8 fatty alcohols, C8/C10 fatty alcohols, C10/C12 fatty alcohols, C12/C14 fatty alcohols, C12/C18 fatty alcohols, and most preferably those technical fatty alcohol cuts that are of plant origin. The index n denotes a number between 1 and 20, preferably between 1 and 10, more preferably between 1 and 5, and most preferably between 1 and 3. The counterion for the cationic charge, A⁻, is a physiologically compatible anion, such as halide, methosulfate, phosphate, citrate, tartrate and the like. The counterion is preferably a halide, such as fluoride, chloride, bromide or methosulfate. Most preferably, the anion is chloride.

Particularly preferred examples of the cationic alkyl oligoglucosides are the compounds with the INCI names Laurdimoniumhydroxypropyl Decylglucosides Chloride, Laurdimoniumhydroxypropyl Laurylglucosides Chloride, Stearyldimoniumhydroxypropyl Decylglucosides Chloride, Stearyldimoniumhydroxypropyl Laurylglucosides Chloride, Stearyldimoniumhydroxypropyl Laurylglucosides Chloride or Cocoglucosides Hydroxypropyltrimonium Chloride.

Such compounds can be obtained from Colonial Chemical Inc. under the designation Suga® Quat, for example.

This also covers, of course, that multiple mixtures of cationic alkyl oligoglucosides can be used. It is preferred in this case if a long-chain and a short-chain cationic alkyl oligoglucoside are used simultaneously.

A further preferred cationic, conditioning polymer comprises at least one structural unit of formula (I), at least one structural unit of formula (II), at least one structural unit of formula (III), and at least one structural unit of formula (IV),

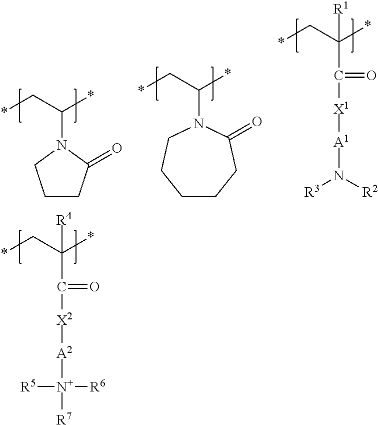

where
R¹ and R⁴, independently of one another, denote a hydrogen atom or a methyl group;
X¹ and X², independently of one another, denote an oxygen atom or an NH group;
A^{l} and A², independently of one another, denote an ethane-1,2-diyl, propane-1,3-diyl or butane- 1,4-diyl group;
R², R³, R⁵ and R⁶, independently of one another, denote a (C₁ to C₄) alkyl group; and R⁷ denotes a (Cs to Cm) alkyl group.

According to the above formulas and all formulas provided hereafter, a chemical bond identified by the “*” symbol denotes a free valence of the corresponding structure fragment.

To compensate for the positive polymer charge in the agent, all possible physiologically compatible anions may be used, such as chloride, bromide, bisulfate, methyl sulfate, ethyl sulfate, tetrafluoroborate, phosphate, hydrogen phosphate, dihydrogen phosphate, or p-toluenesulfonate, triflate.

Examples of (C₁ to C₄) alkyl groups according to the invention are methyl, ethyl, propyl, isopropyl, butyl, sec-butyl, isobutyl, tert-butyl.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl).

The following cationic, conditioning polymers may be used in the agents if the conditioning polymers with respect to the above-mentioned formulas (I) to (IV) meet one or more of the following features:
R¹ and R⁴ each denote a methyl group;
X¹ denotes an NH group;
X² denotes an NH group;
A¹ and A², independently of one another, denote ethane-1,2-diyl or propane-1,3-diyl;
R², R³, R⁵ and R⁶, independently of one another, denote methyl or ethyl (particularly preferably methyl);
R⁷ denotes a (C₁₀ to C₂₄) alkyl group, in particular decyl (caprinyl), dodecyl (lauryl), tetradecyl (myristyl), hexadecyl (cetyl), octadecyl (stearyl), eicosyl (arachyl), or docosyl (behenyl).

It is preferable to select the structural unit of formula (III) from at least one structural unit of formulas (III-1) to (III-8).

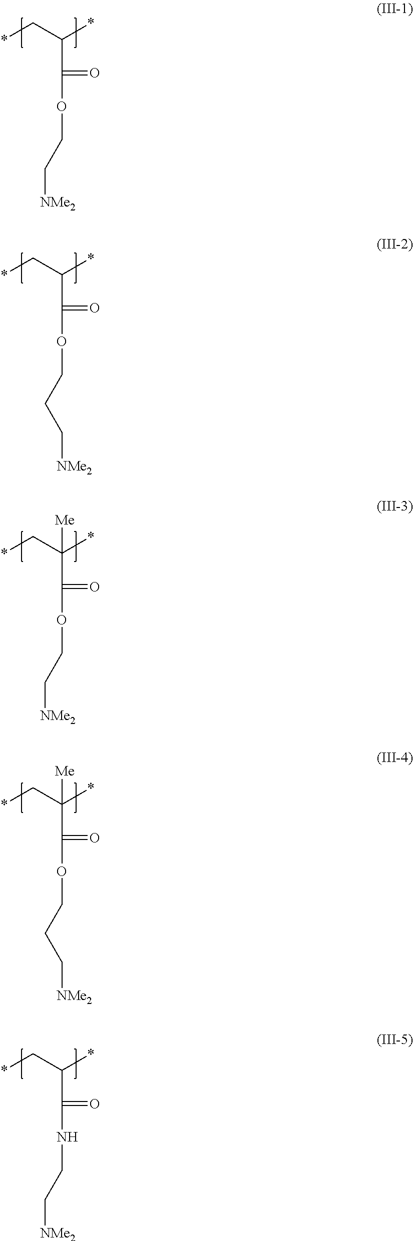

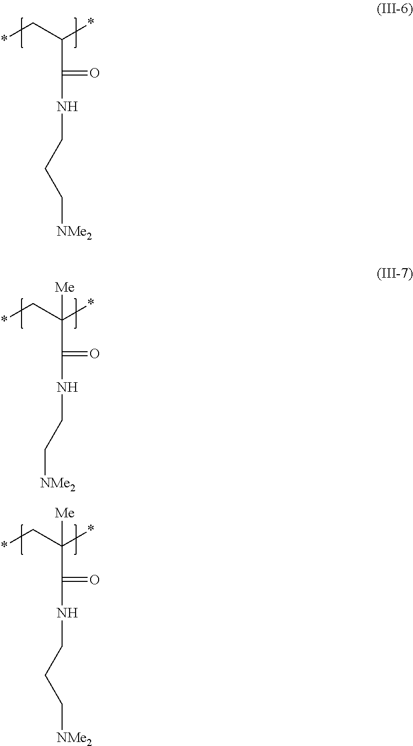

Moreover, it proved to be particularly preferred to select the structural units according to formula (III-7) and/or formula (III-8) as the structural unit of formula (III). The structural unit of formula (III-8) is an especially particularly preferred structural unit.

Furthermore, with respect to achieving the object, it was found to be preferable if the structural unit of formula (IV) is selected from at least one structural unit of formula (IV-1) to (IV-8),

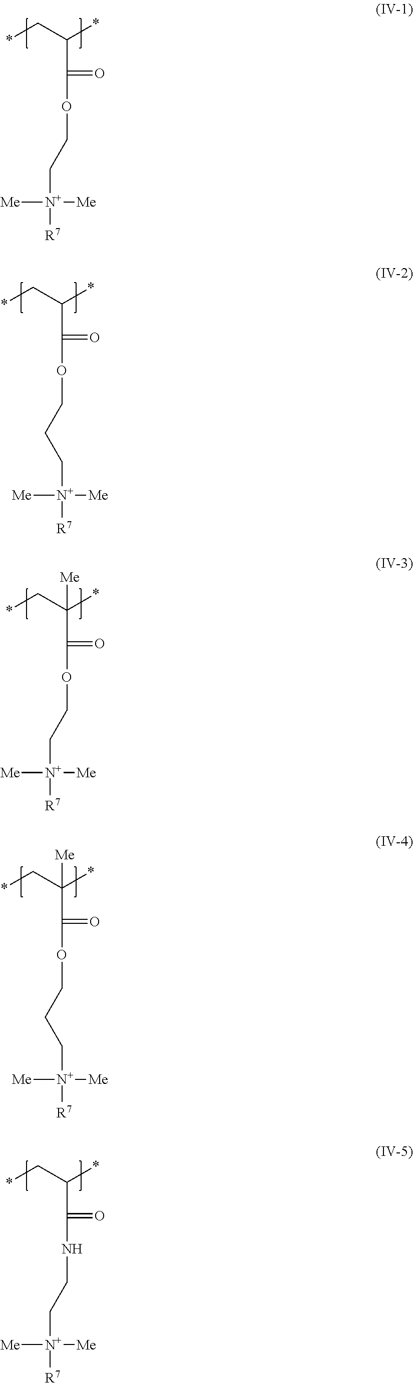

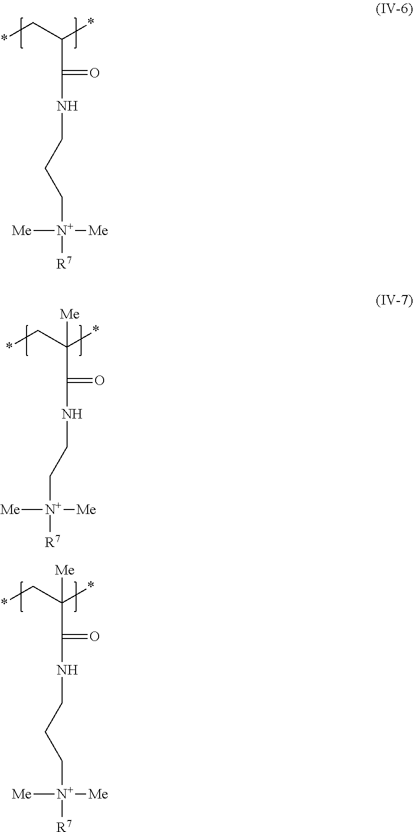

where R⁷ denotes a (C₈ to C₃₀) alkyl group.

Once again, particularly preferred structural units of formula (IV) are the structural units of formula (IV-7) and/or of formula (IV-8), where R⁷ in each case denotes octyl (capryl), decyl (caprinyl), dodecyl (lauryl), tetradecyl (myristyl), hexadecyl (cetyl), octadecyl (stearyl), eicosyl (arachyl), or docosyl (behenyl). The structural unit of formula (IV-8) constitutes an especially particularly preferred structural unit of formula (IV).

An especially particularly preferred cationic, conditioning polymer that is present in the agent comprises at least one structural unit of formula (I), at least one structural unit of formula (II), at least one structural unit of formula (III-8), and at least one structural unit of formula (IV-8),

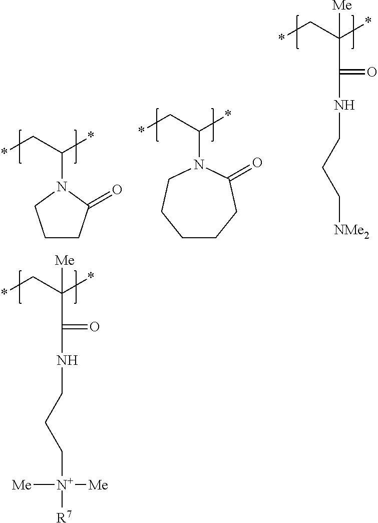

where R⁷ denotes octyl (capryl), decyl (caprinyl), dodecyl (lauryl), tetradecyl (myristyl), hexadecyl (cetyl), octadecyl (stearyl), eicosyl (arachyl), or docosyl (behenyl).

An especially particularly preferred cationic, conditioning polymer is the co-polymer of N-vinylpyrrolidone, N-vinylcaprolactam, N-(3-dimethylaminopropyl)methacrylamide and 3 -(methacryloylamino)propyl-lauryl-dimethylammonium chloride (INCI name: Polyquaternium-69), which is sold by ISP, for example, under the trade name AquaStyle° 300 (28 to 32 wt. % active substance in an ethanol-water mixture, molecular weight 350000).

Further preferred cationic, conditioning polymers are, for example:
cationized honey, for example the commercial product Honeyquat® 50;
polymeric dimethyl diallyl ammonium salts and the copolymers thereof with esters and amides of acrylic acid and methacrylic acid. The products available commercially under the designations Merquat° 100 (poly(dimethyldiallylammonium chloride)) and Merquat° 550 (dimethyldiallylammonium chloride/acrylamide copolymer) are examples of such cationic, conditioning polymers with the INCI name Polyquaternium-7; vinylpyrrolidone/vinylimidazolium methochloride copolymers, as they are offered under the designations Luviquat° FC 370, FC 550 and the INCI name Polyquaternium-16, as well as FC 905 and HM 552;
quaternized polyvinyl alcohol;
and the polymers known under the designations Polyquaternium-2, Polyquaternium-17, Polyquaternium-18, and Polyquaternium-27, comprising quaternary nitrogen atoms in the polymer main chain; and
vinylpyrrolidone-vinylcaprolactam-acrylate terpolymers, as they are commercially offered, with acrylic acid esters and acrylic acid amides serving as the third monomer unit, under the designation Aquaflex° SF 40, for example.

Suitable amphoteric, conditioning polymers are those polymers in which a cationic group derives from at least one of the following monomers:
(i) monomers with quaternary ammonium groups of general formula (Mono1),

R¹—CH═CR²—CO—Z—(CₙH₂ₙ)—N⁽⁺⁾R²R³R⁴ A⁽⁻⁾ (Mono1)

in which R¹ and R², independently of one another, denote hydrogen or a methyl group, R³, R⁴ and R⁵, independently of one another, denote alkyl groups having 1 to 4 carbon atoms, Z denotes an NH group or an oxygen atom, n is an integer from 2 to 5, and A⁽⁻⁾ represents the anion of an organic or inorganic acid;
(ii) monomers with quaternary ammonium groups of general formula (Mono2),

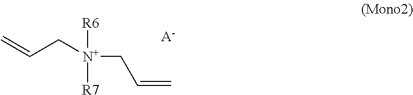

where R⁶ and R⁷, independently of one another, denote a (C₁ to C₄) alkyl group, and in particular a methyl group; and
A⁻ is the anion of an organic or inorganic acid;
(iii) monomeric carboxylic acids of general formula (Mono3)

R⁸—CH═CR⁹—COOH (Mono3)

in which R⁸ and R⁹, independently of one another, are hydrogen or methyl groups.

Particularly preferred are those polymers in which monomers of type (i) are used, in which R³, R⁴ and R⁵ are methyl groups, Z is an NH group, and A⁽⁻⁾ is a halide, methoxysulfate or ethoxysulfate ion; acrylamidopropyl trimethylammonium chloride is a particularly preferred monomer (i). The monomer (ii) used for the described polymer products is preferably acrylic acid.

Particularly preferred amphoteric, conditioning polymers are copolymers of at least one monomer (Mono1) or (Mono2) with the monomer (Mono3), and in particular copolymers of the monomers (Mono2) and (Mono3). Especially particularly preferred amphoteric polymers are copolymer products of diallyl dimethylammonium chloride and acrylic acid. These copolymers are sold under the INCI name Polyquaternium-22, among other things under the trade name Merquat° 280 (ex Lubrizol).

In addition to a monomer (Mono 1) or (Mono2) and a monomer (Mono3), the amphoteric, conditioning polymers may also comprise a monomer (Mono4)
(I) monomeric carboxamides of general formula (Mono4),

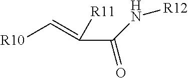

in which R¹⁰ and R¹¹, independently of one another, denote hydrogen or methyl groups, and R¹² denotes a hydrogen atom or a (C₁ to C₈) alkyl group.

Especially particularly preferred amphoteric, conditioning polymers based on comonomers (Mono4) are terpolymers of diallyl dimethylammonium chloride, acrylamide and acrylic acid. These copolymer products are sold under the INCI name Polyquaternium-39, among other things under the trade name Merquat° Plus 3330 (ex Lubrizol).

The amphoteric, conditioning polymers can generally either be used directly or in salt form, which is obtained by neutralization of the polymer product, for example with an alkali hydroxide.

The above-described polymers constitute only some of the conditioning polymers that may be used. So as not to have to describe all suitable cationic and/or amphoteric polymers, including the compositions thereof, a summary of the INCI names of the preferred polymers is provided. The preferred polymers bear the following INCI names: Polyquaternium-2, Polyquaternium-4, Polyquaternium-6, Polyquaternium-7, Polyquaternium-10, Polyquaternium-11, Polyquaternium-15, Polyquaternium-16, Polyquaternium-17, Polyquaternium-18, Polyquaternium-22, Polyquaternium-24, Polyquaternium-28, Polyquaternium-32, Polyquaternium-33, Polyquaternium-34, Polyquaternium-35, Polyquaternium-37, Polyquaternium-39, Polyquaternium-41, Polyquaternium-42, Polyquaternium-44, Polyquaternium-46, Polyquaternium-47, Polyquaternium-55, Polyquaternium-67, Polyquaternium-68, Polyquaternium-69, Polyquaternium-72, Polyquaternium-74, Polyquaternium-76, Polyquaternium-86, Polyquaternium-89, Polyquaternium-95 and Polyquaternium-101, and the mixtures thereof.

It is preferred for the conditioning polymer to be selected from the group consisting of cationic guar compounds, poly(methacryloyloxyethyl trimethylammonium chloride), polymers known under the INCI names Polyquaternium-11, Polyquaternium-46 and Polyquaternium-72, and mixtures thereof

Guar is a polysaccharide obtained from the seeds of the guar bean. Cationic guar compounds are usually quaternized guar compounds, which are obtained by reacting the polysaccharide with trimethylammonium-substituted epoxides.

Suitable cationic guar compounds are sold under the trade name Jaguar and have the INCI name Guar Hydroxypropyltrimonium Chloride. Moreover, particularly suited cationic guar compounds are also commercially available from Hercules under the designation ^{NHance}®^{.} Further cationic guar compounds are sold by BASF SE under the designation ^{Cosmedia}®^{.} A preferred cationic guar compound is the commercial product AquaCat° from Hercules. This raw material is an already pre-dissolved cationic guar compound.

Poly(methacryloyloxyethyl trimethylammonium chloride), which may also be cross-linked, is a homopolymer known under the INCI name Polyquaternium-37. Such products are commercially available, for example, under the designations Cosmedia° CTH or Cosmedia® Ultragel 300 (BASF SE) or Synthalen® CR (3V Group).

The homopolymer is preferably used in the form of a non-aqueous polymer dispersion. Such polymer dispersions are commercially available under the designations Salcare° SC 95 and Salcare° SC 96. A polymer dispersion sold under the designation Cosmedia® Triple C (ex BASF SE) is likewise suited.

Polyquaternium-11 is the reaction product of diethyl sulfate with a copolymer of vinylpyrrolidone and dimethylaminoethyl methacrylate. Suitable commercial products are available, for example, under the designations Dehyquart® CC 11 and Luviquat® PQ 11 PN from BASF SE, or Gafquat 440, Gafquat 734, Gafquat 755 or Gafquat 755N from Ashland Inc. Polyquaternium-11 is preferably used in a quantity of 1 to 10 wt. %, based on the total weight of the cosmetic composition.

Polyquaternium-46 is the reaction product of vinylaprolactam and vinylpyrrolidone with methylvinylimidazolium methosulfate and is available from BASF SE, for example, under the designation Luviquat® Hold. Polyquaternium-46 is preferably used in a quantity of 1 to 5 wt. %, based on the total weight of the cosmetic composition.

A particularly preferred cationic, conditioning polymer is Polyquaternium-72. Polyquaternium-72 is a special cellulose. This cellulose is a hydroxyethyl cellulose that was quaternized. Polyquaternium-72 is a trimonium and cocodimonium hydroxyethyl cellulose. Polyquaternium-72 may be used either in solid form or already pre-dissolved in an aqueous solution. Use of the commercial product Mirustyle® CP from Croda, for example, is preferred. Polyquaternium-72 is preferably present in the cosmetic compositions in a quantity of at least 0.5 wt. %, based on the total weight of the cosmetic compositions. Polyquaternium-72 is preferably used in a quantity of 1 to 10 wt. %, based on the total weight of the cosmetic composition.

Of all the above-mentioned conditioning polymers, Polyquaternium-72 is preferably used as the conditioning polymer.

The total amount of conditioning polymer, based the total weight of the cosmetic composition, is preferably 0.1 to 10 wt. %, more preferably 1 to 9 wt. %, and in particular preferably 2.5 to 7.5 wt. %.

The cosmetic composition comprises, based on the total weight thereof, a thickening polymer as a second essential component. The cosmetic composition can also comprise several thickening polymers. Thickening polymers shall be understood to mean polymers that increase the viscosity of the cosmetic composition.

Preferably, a polymer known under the INCI name Polyacrylate-1 Crosspolymer is used as the thickening polymer. Polyacrylate-1 Crosspolymer is a copolymer of one or more simple esters of acrylic or methacrylic acid, C1-4 dialkylamino C1-6 alkyl methacrylate, PEG/PPG-30/5 allyl ether, PEG 20-25 C10-30 alkyl ether methacrylate, hydroxyl C2-6 alkyl methacrylate cross-linked with ethylene glycol dimethacrylate. Carbopol Aqua CC (ex Lubrizol/Noveon) is a particularly preferred Polyacrylate-1 Crosspolymer.

The cosmetic compositions comprise the thickening polymer especially in amounts of 0.5 to 25 wt. %, preferably in amounts of 5 to 20 wt. %, and especially particularly preferably in amounts of 12.5 to 17.5 wt. %, in each case based on the total weight of the compositions.

The cosmetic composition comprises, based on the total weight thereof, a film-forming polymer as a third essential component. The cosmetic composition can also comprise several film-forming polymers. Film-forming polymers shall be understood to mean polymers that, on drying, leave behind a continuous film on the hair. Film-forming polymers are furthermore understood to include polymers that, when used in a 0.01 to 20 wt. % aqueous, alcoholic or aqueous-alcoholic solution, are capable of depositing a transparent polymer film on the hair.

Permanently and temporarily cationic, anionic, non-ionic or amphoteric polymers are suitable film-forming polymers. The film-forming polymers may be of synthetic or natural origin.

Suitable preferred synthetic, film-forming polymers are homopolymers or copolymers composed of at least one of the following monomers: vinylpyrrolidone, vinylcaprolactam, vinyl ester such as vinyl acetate, vinyl alcohol, acrylamide, methacrylamide, C₁ to C₇ alkyl acrylamide, C₁ to C₇ dialkyl acrylamide, C₁ to C₇ alkyl methacrylamide, C₁ to C₇ dialkyl methacrylamide, C₁ to C₇ alkyl acrylate, acrylic acid, propylene glycol, ethylene glycol, wherein the C₁ to C₇ alkyl groups of these monomers are preferably C₁ to C₃ alkyl groups. Homopolymers of vinylcaprolactam, of vinylpyrrolidone or of N-vinylformamide shall be mentioned by way of example. Further suitable synthetic film-forming, hair-setting polymers are, for example, copolymer products of vinylpyrrolidone and vinyl acetate, vinylpyrrolidone and styrene, or terpolymers of vinylpyrrolidone, vinyl acetate and vinyl propionate, polyacrylamides. Suitable natural film-forming polymers are, for example, cellulose derivatives, such as hydroxypropyl cellulose having a molecular weight of 30,000 to 50,000 g/mol.

Further examples of common film-forming polymers include acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C1-2 succinates/hydroxyacrylates copolymer, acrylates/lauryl acrylate/stearyl acrylate/ethylamine oxide methacrylate copolymer, acrylates/octylacrylamide copolymer, acrylates/octyl acryl amide/diphenyl aodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol-acrylates/acrylamide copolymer, amino ethyl propanediol-AMPD-acrylates/diacetoneacrylamide copolymer, ammonium VA/acrylates copolymer, AMPD-acrylates/diacetoneacrylamide copolymer, AMP-acrylates/allyl methacrylate copolymer, AMP-acrylates/C1-18 alkyl acrylates/C1-8 alkyl acrylamide copolymer, AMP-acrylates/diacetoneacrylamide copolymer, AMP-acrylates/dimethylaminoethylmethacrylate copolymer, Bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, corn starch/acrylamide/sodium acrylate copolymer, diethylene glycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MA copolymer, isobutylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, polybeta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, Polysilicone-9, Polyurethane-1, Polyurethane-6, Polyurethane-10, polyvinyl acetate, polyvinyl butyral, polyvinylcaprolactam, polyvinylformamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVP/VA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethyl amino ethylmethacryl ate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

The film-forming polymer is preferably a non-ionic, film-forming polymer. It is in particular preferred for the cosmetic compositions to comprise polyvinylpyrrolidone (PVP) and/or a vinylpyrrolidone-containing copolymer as the film-forming polymer. Suitable polyvinylpyrrolidones are available, for example, from BASF SE under the designation Luviskol® K. Of the vinylpyrrolidone-containing copolymers, especially particularly preferably a styrene/VP copolymer and/or a vinylpyrrolidone/vinyl acetate copolymer and/or a VP/DMAPA acrylate copolymer and/or a VP/vinylcaprolactam/DMAPA acrylates copolymer are used in the cosmetic compositions.

Vinylpyrrolidone/vinylacetate copolymers are sold by BASF SE under the designation Luviskol® VA. A VP/vinylcaprolactam/DMAPA acrylates copolymer is sold by Ashland, Inc., for example, under the trade name Aquaflex® SF-40. A VP/DMAPA acrylates copolymer is sold by Ashland, for example, under the designation Styleze CC-10 and is a most preferred vinylpyrrolidone-containing copolymer.

In particular when poly(methacryloyloxyethyl trimethylammonium chloride) is used as the conditioning polymer, it may be preferred for the cosmetic composition to comprise polyvinylpyrrolidone and VP/DMAPA acrylates copolymer as the film-forming polymer.

The cosmetic compositions comprise the film-forming polymer especially in amounts of 0.1 to 30 wt. %, preferably in amounts of 1 to 25 wt. %, and especially particularly preferably in amounts of 5 to 20 wt. %, in each case based on the total weight of the compositions.

The cosmetic composition comprises at least one humectant as a fourth essential component.

The humectant is preferably selected from the group consisting of polyols, polyether-modified polysiloxanes and mixtures thereof

The humectants used are preferably polyether-modified polysiloxanes of the following formula (I):

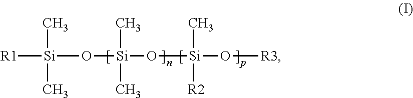

in which—groups R1 and R3, independently of one another, denote a hydrogen atom, an alkyl group having 1 to 30 carbon atoms, an alkoxy group having 1 to 30 carbon atoms or an optionally substituted phenyl group;
group R2 denotes the group —C_{c}H_{2c}—O—(C₂H₄O—)ₐ(C₃H₆O—)_{b}R5;
group R5 denotes a hydrogen atom or a linear or branched alkyl group having 1 to 16 carbon atoms;
n denotes a number from 0 to 500;
p denotes a number from 1 to 50;
a denotes a number from 0 to 50;
b denotes a number from 0 to 50;
a+b is at least 1, and
c denotes a number from 1 to 4.

More preferred polyether-modified polysiloxane compounds of the general chemical formula (I) are:

| | R1, R3 | R2 | R5 | n | p | a | b | c |
|---|---|---|---|---|---|---|---|---|
| 1 | H, methyl | —C_{c}H_{2c}—O—(C₂H₄O)ₐ—(C₃H₆O)_{b}—R5 | H, methyl | 1-500 | 5-50 | 15-25 | 15-25 | 3 |
| 2 | H, methyl | —C_{c}H_{2c}—O—(C₂H₄O)ₐ—(C₃H₆O)_{b}—R5 | H, methyl | 1-500 | 5-50 | 5-20 | 0.1 | 3 |
| 3 | H, methyl | —C_{c}H_{2c}—O—(C₂H₄O)ₐ—(C₃H₆O)_{b}—R5 | H, methyl | 0.1 | 1-3 | 5-10 | 0.1 | 2 |
| 4 | H, methyl | —C_{c}H_{2c}—O—(C₂H₄O)ₐ—(C₃H₆O)_{b}—R5 | H, methyl | 1-500 | 5-50 | 20-30 | 20-30 | 3 |
| 5 | H, methyl | —C_{c}H_{2c}—O—(C₂H₄O)ₐ—(C₃H₆O)_{b}—R5 | H, methyl | 1-500 | 5-50 | 10-25 | 10-25 | 3 |
| 6 | H, methyl | —C_{c}H_{2c}—O—(C₂H₄O)ₐ—(C₃H₆O)_{b}—R5 | H, methyl | 1-500 | 2-50 | 10-30 | 2-10 | 3 |
| 7 | H, methyl | —C_{c}H_{2c}—O—(C₂H₄O)ₐ—(C₃H₆O)_{b}—R5 | H, methyl | 1-500 | 2-50 | 10-20 | 1-10 | 3 |

Particularly preferred polyether-modified polysiloxane compounds of the general chemical formula (I) are:

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R5 | H, methyl | 5-500 | 7-50 | 15-20 | 15-20 | 3 |
| 2 | Methyl | —C_{c}H_{2c}—O—(C₂H₄O)ₐ—(C₃H₆O)_{b}—R5 | H, methyl | 5-500 | 7-50 | 7-15 | 0 | 3 |
| 3 | Methyl | —C_{c}H_{2c}—O—(C₂H₄O)ₐ—(C₃H₆O)_{b}—R5 | H, methyl | 0.1 | 1 | 6-8 | 0 | 2 |
| 4 | Methyl | —C_{c}H_{2c}—O—(C₂H₄O)ₐ—(C₃H₆O)_{b}—R5 | H, methyl | 5-500 | 7-50 | 20-25 | 20-25 | 3 |
| 5 | Methyl | —C_{c}H_{2c}—O—(C₂H₄O)ₐ—(C₃H₆O)_{b}—R5 | H, methyl | 5-500 | 7-50 | 15-20 | 15-20 | 3 |
| 6 | Methyl | —C_{c}H_{2c}—O—(C₂H₄O)ₐ—(C₃H₆O)_{b}—R5 | H, methyl | 5-500 | 2-50 | 15-25 | 2-8 | 3 |
| 7 | Methyl | —C_{c}H_{2c}—O—(C₂H₄O)ₐ—(C₃H₆O)_{b}—R5 | H, methyl | 5-500 | 2-50 | 10-15 | 1-5 | 3 |

In particular, preferred polyether-modified polysiloxane compounds of the general chemical formula (I) are:

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R5 | Methyl | 10-500 | 10-50 | 18 | 18 | 3 |
| 2 | Methyl | —C_{c}H_{2c}—O—(C₂H₄O)ₐ—(C₃H₆O)_{b}—R5 | Methyl | 10-500 | 10-50 | 12 | 0 | 3 |
| 3 | Methyl | —C_{c}H_{2c}—O—(C₂H₄O)ₐ—(C₃H₆O)_{b}—R5 | Methyl | 10-500 | 10-50 | 14 | 0 | 3 |
| 4 | Methyl | —C_{c}H_{2c}—O—(C₂H₄O)ₐ—(C₃H₆O)_{b}—R5 | Methyl | 0 | 1 | 7 | 0 | 2 |
| 5 | Methyl | —C_{c}H_{2c}—O—(C₂H₄O)ₐ—(C₃H₆O)_{b}—R5 | Methyl | 10-500 | 10-50 | 22 | 24 | 3 |
| 6 | Methyl | —C_{c}H_{2c}—O—(C₂H₄O)ₐ—(C₃H₆O)_{b}—R5 | Methyl | 10-500 | 10-50 | 17 | 18 | 3 |
| 7 | Methyl | —C_{c}H_{2c}—O—(C₂H₄O)ₐ—(C₃H₆O)_{b}—R5 | Methyl | 10-500 | 5-50 | 20 | 6 | 3 |
| 8 | Methyl | —C_{c}H_{2c}—O—(C₂H₄O)ₐ—(C₃H₆O)_{b}—R5 | Methyl | 10-500 | 5-50 | 14 | 4 | 3 |

Polyether-modified polysiloxane compounds listed in above Tables 1 to 3 are commercially available, for example under the following trade names:
1: Dow Corning 190 (INCI: PEG/PPG-18/18 Dimethicone),
2: Dow Corning 193 (INCI: PEG-12 Dimethicone),
3: ABIL® B 8843 (INCI: PEG-14 Dimethicone),
4: Silwet L-77,
5: Mirasil DCMO (INCI: Cyclomethicone, PEG/PPG-22/24 Dimethicone),
6: Dow Corning Q2-5220 (INCI: PEG/PPG-17/18 Dimethicone),
7: ABIL® B 88184 (INCI: PEG/PPG-20/6 Dimethicone), and
8: ABIL® B 8851 (INCI: PEG/PPG-14/4 Dimethicone).

In particular, preferred cosmetic compositions are characterized by comprising at least one polyether-modified polysiloxane compound according to formula (I) that meets the conditions of line 2 (Tables 1-3) (for example Dow Corning 193 (INCI: PEG-12 Dimethicone)).

Likewise in particular preferred cosmetic compositions are characterized by comprising at least one polyether-modified polysiloxane compound according to formula (I) that meets the conditions of line 2 in Tables 1 and 2 and of line 3 in Table 3 (for example ABIL® B 8843 (INCI: PEG-14 Dimethicone)).

The cosmetic compositions can comprise a polyol as the humectant.

The polyol is preferably an organic polyol and may be used alone or in the form of polyol mixtures. Preferred cosmetic agents are characterized by comprising less than four, especially one to three, in particular, however, only one or two polyols. In particular, polyols from the group consisting of glycerol, 1,2-ethanediol, polyethylene glycols having a MW >400, propanediol, butanediol, in particular 1,3-butanediol, hexanediol, in particular 1,6-hexanediol, sorbitol, threitol, erythritol, arabitol, altritol, ribitol, xylitol, galactitol, mannitol, iditol and panthenol are suitable for producing cosmetic compositions. Preferred cosmetic compositions are characterized by comprising at least one polyol from the group consisting of glycerol, sorbitol, panthenol, polyethylene glycol, propylene glycol, butylene glycol and hexanediol as the organic polyol. It is especially particularly preferred for the cosmetic composition at least three compounds selected from the group consisting of polyether-modified polysiloxane, which is known under the INCI name PEG-12 Dimethicone, glycerol, panthenol, propylene glycol and hexanediol.

Adding a polyol can enhance the properties of the cosmetic compositions during storage and application and has beneficial cosmetic effects.

The cosmetic compositions comprise the humectant especially in amounts of 1 to 35 wt. %, preferably in amounts of 5 to 30 wt. %, and especially particularly preferably in amounts between 10 to 25 wt. %, in each case based on the total weight of the compositions.

For balancing purposes, a smaller amount of water is preferably added to the compositions if cosmetic compositions have a high polyol content.

The cosmetic composition comprises at least one acid as a fifth essential component. The acid used may be an inorganic acid, such as hydrochloric acid, or an organic acid. Use of an organic acid is preferred. It is furthermore preferred for the acid to be selected from the group consisting of formic acid, acetic acid, glycolic acid, gluconic acid, lactic acid, mandelic acid, citric acid, glyoxalic acid and mixtures thereof. The use of lactic acid is particularly preferred in the cosmetic compositions.

The cosmetic compositions comprise the acid especially in amounts of 0.1 to 6 wt. %, preferably in amounts of 0.25 to 4 wt. %, and especially particularly preferably in amounts of 0.5 to 2.5 wt. %, in each case based on the total weight of the compositions.

Further components of the cosmetic compositions are described hereinafter, which may be present in the compositions in addition to the essential ingredients described above.

Further suitable ingredients include surfactants, fats, waxes, protein hydrolysates, amino acids, oligopeptides, vitamins, provitamins, vitamin precursors, betaines, bioquinones, purine (derivatives), taurine (derivatives), plant extracts, silicones, ester oils, UV sunscreens, structurants, thickeners, electrolytes, pH-setting agents, swelling agents, dyes, anti-dandruff active ingredients, complexing agents, opacifiers, pearlizing agents, pigments, stabilizers, propellants, antioxidants, perfume oils and/or preservatives.

The cosmetic agents preferably also comprise at least one representative from the group of the anionic, amphoteric, zwitterionic, non-ionic, cationic surfactants or the mixtures thereof. It is preferred to use solid emulsifiers at room temperature (20° C.), and in particular to use solid non-ionic emulsifiers at room temperature (20° C.).

In particular, the fatty acid partial glycerides, which shall be understood to mean monoglycerides, diglycerides and the technical mixtures thereof, are suitable non-ionic emulsifiers. Typical examples are monoglycerides and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof. The use of oleic acid monoglycerides are preferred. Another group of preferred emulsifiers is the surface-active, medium-chain fatty alcohols, in particular cetyl alcohol and stearyl alcohol.

The use of at least one cationic surfactant selected from quaternary ammonium compounds, the ester quats, the amidoamines or mixtures thereof is preferred. Preferred quaternary ammonium compounds are ammonium halides, in particular chlorides and bromides, such as alkyl trimethylammonium chlorides, dialkyldimethylammonium chlorides and trialkylmethylammonium chlorides. The long alkyl chains of these surfactants preferably comprise 10 to 18 carbon atoms, such as in cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. Further preferred cationic surfactants are the imidazolium compounds known under the INCI names Quaternium-27 and Quaternium-83.

The additional surfactants are preferably present in the compositions an amount of 0.01 wt. % to 5 wt. %, and particularly preferably of 0.1 wt. % to 2.5 wt. %, in each case based on the weight of the composition.

The water content of the cosmetic compositions is at least 1 wt. %, preferably at least 5 wt. %, and in particular preferably between 10 and 70 wt. %, in each case based on the total weight of the cosmetic composition.

Preferred cosmetic compositions are characterized by the following:
a) 0.1 to 10 wt. % of a conditioning polymer selected from the group consisting of cationic guar compounds, poly(methacryloyloxyethyl trimethylammonium chloride), polymers known under the INCI names Polyquaternium-11, Polyquaternium-46 and Polyquaternium-72, and mixtures thereof;
b) 0.5 to 25 wt. % of a thickening polymer comprising a compound known under the INCI name Polyacrylate-1 Crosspolymer;
c) 0.1 to 30 wt. % of a film-forming polymer selected from the group consisting of polyvinylpyrrolidone (PVP), a vinylpyrrolidone-containing copolymer and mixtures thereof;
d) 1 to 35 wt. % of a humectant selected from the group consisting of polyols, polyether-modified polysiloxanes and mixtures thereof;
e) 0.1 to 6 wt. % of an organic acid, and
f) 1 to 70 wt. % water.

More preferred cosmetic compositions are furthermore characterized by the following:
a) 0.1 to 10 wt. % of a conditioning polymer selected from the group consisting of cationic guar compounds, poly(methacryloyloxyethyl trimethylammonium chloride), polymers known under the INCI names Polyquaternium-11, Polyquaternium-46 and Polyquaternium-72, and mixtures thereof;
b) 0.5 to 25 wt. % of a thickening polymer comprising a compound known under the INCI name Polyacrylate-1 Crosspolymer;
c) 0.1 to 30 wt. % of a film-forming polymer comprising polyvinylpyrrolidone and VP/DMAPA acrylates copolymer;
d) 1 to 35 wt. % of a humectant selected from the group consisting of polyether-modified polysiloxane, which is known under the INCI name PEG-12 Dimethicone or PEG-14 Dimethicone, glycerol, sorbitol, panthenol, polyethylene glycol, propylene glycol, butylene glycol, hexanediol and mixtures thereof;
e) 0.1 to 6 wt. % of an organic acid to be selected from the group consisting of formic acid, acetic acid, glycolic acid, gluconic acid, lactic acid, mandelic acid, citric acid, glyoxalic acid and mixtures thereof, and
f) 1 to 70 wt. % water.

Still more preferred cosmetic compositions are characterized by the following:
a) 1 to 9 wt. % of a conditioning polymer selected from the group consisting of cationic guar compounds, poly(methacryloyloxyethyl trimethylammonium chloride), polymers known under the INCI names Polyquaternium-11, Polyquaternium-46 and Polyquaternium-72, and mixtures thereof;
b) 5 to 20 wt. % of a thickening polymer comprising a compound known under the INCI name Polyacrylate-1 Crosspolymer;
c) 1 to 25 wt. % of a film-forming polymer comprising polyvinylpyrrolidone and VP/DMAPA acrylates copolymer;
d) 5 to 30 wt. % of a humectant selected from the group consisting of polyether-modified polysiloxane, which is known under the INCI name PEG-12 Dimethicone or PEG-14 Dimethicone, glycerol, sorbitol, panthenol, polyethylene glycol, propylene glycol, butylene glycol, hexanediol and mixtures thereof;
e) 0.25 to 4 wt. % of an organic acid to be selected from the group consisting of formic acid, acetic acid, glycolic acid, gluconic acid, lactic acid, mandelic acid, citric acid, glyoxalic acid and mixtures thereof, and
f) 1 to 70 wt. % water.

Even still more preferred cosmetic compositions are characterized by the following:
a) 2.5 to 7.5 wt. % of a conditioning polymer selected from the group consisting of cationic guar compounds, poly(methacryloyloxyethyl trimethylammonium chloride), polymers known under the INCI names Polyquaternium-11, Polyquaternium-46 and Polyquaternium-72, and mixtures thereof;
b) 12.5 to 17.5 wt. % of a thickening polymer comprising a compound known under the INCI name Polyacrylate-1 Crosspolymer;
c) 5 to 20 wt. % of a film-forming polymer comprising polyvinylpyrrolidone and VP/DMAPA acrylates copolymer;
d) 10 to 25 wt. % of a humectant selected from the group consisting of polyether-modified polysiloxane, which is known under the INCI name PEG-12 Dimethicone or PEG-14 Dimethicone, glycerol, sorbitol, panthenol, polyethylene glycol, propylene glycol, butylene glycol, hexanediol and mixtures thereof;
e) 0.5 to 2.5 wt. % of an organic acid comprising lactic acid, and
f) 10 to 70 wt. % water.

Especially particularly preferred cosmetic compositions are characterized by the following:
a) 2.5 to 7.5 wt. % of a conditioning polymer comprising a polymer known under the INCI name Polyquaternium-72;
b) 12.5 to 17.5 wt. % of a thickening polymer comprising a compound known under the INCI name Polyacrylate-1 Crosspolymer;
c) 5 to 20 wt. % of a film-forming polymer comprising polyvinylpyrrolidone and VP/DMAPA acrylates copolymer;
d) 10 to 25 wt. % of a humectant comprising at least three compounds selected from the group consisting of polyether-modified polysiloxane, which is known under the INCI name PEG-12 Dimethicone, glycerol, panthenol, propylene glycol and hexanediol;
e) 0.5 to 2.5 wt. % of an organic acid comprising lactic acid, and
f) 10 to 70 wt. % water.

A second subject matter of the present invention is the use of a cosmetic composition according to the invention for temporarily reshaping keratin-containing fibers, and in particular human hair. The human hair is most preferably curly human hair.

A third subject matter of the present invention is the use of a cosmetic composition according to the invention for improving at least one of the following properties of curly hair:
curl retention;
bounce of curls;
curl separation;
shine; and
manageability.

The cosmetic compositions according to the invention can be used in a method for temporarily reshaping keratin-containing fibers, and in particular human hair, in which a cosmetic composition is applied to the keratin fibers and the shape of these is temporarily imparted hold. The human hair is most preferably curly human hair. The cosmetic composition is preferably applied to shampooed and towel-dried hair, and the hair is then air-dried or dried using heat, for example an electric hair dryer, a curling iron or a hood dryer, optionally with the aid of re-shaping aids such as rollers or curlpapers.

What was said with respect to the compositions applies, mutatis mutandis, with respect to preferred embodiments of the use or the method.

The make-up of a preferred cosmetic composition A can be derived from the following table (information in percent by weight refers to the total weight of the cosmetic agent, unless indicated otherwise).

| Ingredient | A |
|---|---|
| Cetrimonium chloride | 2 |
| Polyquaternium-72 | 5 |
| PEG-12 Dimethicone | 1 |
| PVP | 1.5 |
| VP/DMAPA Acrylates Copolymer | 5 |
| Glycerol | 7.5 |
| Propylene glycol | 7.5 |
| Hexanediol | 0.15 |
| Perfume | 0.45 |
| PEG-40 Hydrogenated Castor Oil | 0.55 |
| Lactic acid | 1.25 |
| Panthenol | 0.2 |
| Polyacrylate-1 Crosspolymer | 15 |
| Water, preservative and by-products | to make up to 100 |

Formulation A is a transparent composition having pleasant, sensory properties. When applied by hand, it leaves no greasy/slimy sensation on the hand or on the moist hair treated therewith.

Formulation A provides easy manageability, high shine, a healthy appearance and, to the extent that curls are present, a lasting curly appearance to hair treated therewith, without causing hair to become sticky or inflexible.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A transparent cosmetic composition, comprising, based on the total weight thereof,
a) 0.1 to 10 wt. % of a conditioning polymer;
b) 0.5 to 25 wt. % of a thickening polymer;
c) 0.1 to 30 wt. % of a film-forming polymer;
d) 1 to 35 wt. % of a humectant;
e) 0.1 to 6 wt. % of an acid, and
f) 1 to 90 wt. % water.

2. The transparent cosmetic composition according to claim 1, wherein the film-forming polymer is selected from the group consisting of polyvinylpyrrolidone (PVO), a vinylpyrrolidone-containing copolymer, and mixtures thereof

3. The transparent cosmetic composition according to claim 1, wherein the film-forming polymer includes a mixture of polyvinylpyrrolidone (PVP) and a polymer known under the INCI name VP/DMAPA Acrylates Copolymer.

4. The transparent cosmetic composition according to claim 1, wherein the conditioning polymer is selected from the group consisting of cationic guar compounds, poly(methacryloyloxyethyl trimethylammonium chloride), polymers known under the INCI names Polyquaternium-11, Polyquaternium-46 and Polyquaternium-72, and mixtures thereof

5. The transparent cosmetic composition according to claim 4, wherein the conditioning polymer is a compound known under the INCI name Polyquaternium-72.

6. The transparent cosmetic composition according to claim 1, wherein the thickening polymer is a compound known under the INCI name Polyacrylate-1 Crosspolymer.

7. The transparent cosmetic composition according to claim 1, wherein the humectant is selected from the group consisting of polyols, polyether-modified polysilixanes and mixtures thereof

8. The transparent cosmetic composition according to claim 1, wherein the acid is an organic acid selected from the group consisting of formic acid, acetic acid, glycolic acid, gluconic acid, lactic acid, mandelic acid, citric acid, glyoxalic acid and mixtures thereof

9. The transparent cosmetic composition according to claim 1, further including a cationic surfactant.

10. A method for improving at least one of the following properties of curly hair:
curl retention;
bounce of curls;
curl separation;
shine; and
manageability,
the method including applying to the hair the transparent cosmetic composition according to claim 1.
